(19) United States
(12) Patent Application Publication
(10) Pub. No.: US 2010/0217202 A1
(43) Pub. Date: Aug. 26, 2010

(54) Device for achieving hemostasis at site of puncture wound

(76) Inventor: Timothy W.I. Clark, Philadelphia, PA (US)

Correspondence Address: Jack Shuman, 3762 Carmel Drive, Carmel, IN, 46033-4329, US

(21) Appl. No.: 12/378,919

(22) Filed: Feb. 21, 2009

(51) Int. Cl. A61M 5/315; A61M 5/31

(52) U.S. Cl. 604/180; 604/227

(57) ABSTRACT

A device for use by a medical technician to achieve hemostasis at the site of a puncture wound in the skin of a patient, said device being operable by three fingers of one hand of the medical technician.

### BACKGROUND Of THE INVENTION

1. Field of the Invention

This invention pertains to a novel and efficient device for achieving hemostasis at the site of a puncture wound.

More specifically, this invention pertains to such a device as aforesaid operable by three fingers of one hand of a medical technician, e.g. a nephrology nurse.

2. Description of the Prior Art

There are many devices and procedures currently employed in the medical field for achieving hemostasis at the site of a puncture wound resulting, for example, from a dialysis procedure.

Among such prior art devices and procedures are:
(a) a non-woven sponge manually applied directly to the site of the bleeding at the puncture wound;
(b) clamp-type devices around the arm of the patient;
(c) notch-shaped compression pad tightened around the arm of the patient much like an electrical tie.

None of these prior art devices and procedures disclose, nor even remotely suggest, a device operable by only three fingers of one hand of a medical technician, which is an inexpensive, sterile and single-use device.

One of the objects of this invention is to provide an improved efficient device for achieving hemostasis at the site of a puncture wound, usually in the arm of a patient.

Another of the objects of this invention is to provide such a device operable by only three fingers of a medical technician.

Other and further objects of this invention will become apparent by reference to the accompanying specification and drawings, and to the appended claims.

Briefly, I have discovered that the foregoing objects may be attained by providing a footplate having an internally ratcheted cylinder secured thereto in a central position thereon and curved to bear against the arm of a patient, a plunger positioned within the cylinder and having ratchets complementary to the ratchets within the cylinder, the top of the plunger being bifurcated and curved to receive one finger, namely the index finger, of the medical technician, the bottom of the plunger having a rounded contour. In one embodiment of the invention, two curved arms are mounted on the footplate, one on each side of the cylinder, one of the curved arms receiving the thumb of the medical technician and the other of the curved arms receiving the middle finger of the technician. In another embodiment of the invention, only one curved arm is employed receiving the thumb or middle finger of the medical technician and one side of the cylinder receiving the remaining finger. In yet another embodiment of the invention, no such arms are employed, two opposing sides of the cylinder each receiving one of the aforesaid fingers. The ratchets within the cylinder and the external ratchets on the plunger are complementary, whereby the plunger can be moved in only one direction, downwardly toward the puncture wound.

Pads, with adhesive surfaces on both faces, are secured to the bottom of the footplate, and adhere to the skin of the patient when the device is placed in use. In this manner, the device is maintained in position on the skin of the patient.

### DESCRIPTION OF THE DRAWINGS

Referring now to the drawings, in which like numerals represent like parts in the several views:

FIG. 1 represents a view in perspective of one embodiment of the device, showing the plunger in the internally ratcheted cylinder prior to use.

FIG. 2 represents a view in perspective of the device, directed at the bottom thereof, showing the plunger in the internally ratcheted cylinder prior to use.

FIG. 3 represents a view in perspective of the device, showing the plunger fully advanced in the cylinder, the rounded portion at the bottom of the plunger and the compression pad adhering thereto bearing against the puncture wound site.

FIG. 4 represents a view in perspective of an alternate embodiment of the device, showing the plunger in the internally ratcheted cylinder prior to use.

FIG. 5 represents a view in perspective of yet another embodiment of the device, showing the plunger in the internally ratcheted cylinder prior to use.

### DESCRIPTION OF THE PREFERRED EMBODIMENTS

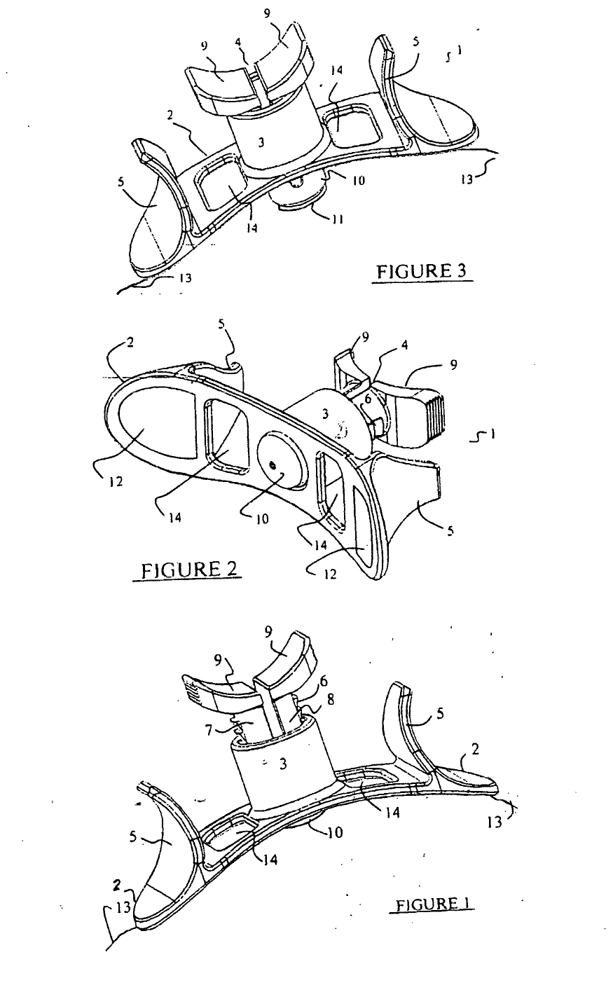

Hemostasis device 1 comprises footplate 2, internally ratcheted cylinder 3 centrally positioned on footplate 2, externally ratcheted plunger 4 and, in the embodiment of FIGS. 1-3, curved arms 5 on footplate 2.

The external ratchets 6 on plunger 4 engage the internal ratchets in cylinder 3 in such manner as to limit plunger 4 to movement downwardly in cylinder 3. In other words, plunger 4 can be forced downwardly toward the puncture wound site, but is restrained from movement upwardly in the other direction in said cylinder 3.

The upper portion of plunger 4 is bifurcated as indicated by the numerals 7 and 8 whereby, due to the resilience of the material from which plunger 4 is made, to force said bifurcations 7 and 8 outwardly against the interior of said cylinder 3, thereby to force external ratchets 6 on plunger 4 into engagement with the internal ratchets in cylinder 3.

The bifurcated portions 7 and 8 at the top of plunger 4 are, at their extreme upper ends, arcuatel shaped, 9, and are adapted to be engaged by the index finger of the medical technician when device 1 is put to use.

The bottom of plunger 4 is provided with a round portion 10 with compression pad 11 adhered thereto. Compression pad 11 may have a pro-coagulant coating such as calcium alginate, oxidized regenerated cellulose, seaweed extracts or a pro-coagulant polymer. Compression pad 11 may also have an antimicrobial coating such as silver or chlorhexidine.

Pads 12, with adhesive surfaces on both faces thereof, are applied to the bottom of footplate 2 and adhere to the skin 13 of the patient when device 1 is in use, thereby to secure the footplate 2 thereto to prevent device 1 from shifting position on the skin 13 when in use.

Curved arms 5 on footplate 2 are engaged by the thumb and middle finger of the medical technician when device 1 is in use.

Footplate 2 is provided with apertures 14 permitting the medical technician to observe the position of the rounded portion 10 of the plunger 4 and the compression pad 11 secured to said rounded portion 10 relative to the site of the puncture wound to assure that the device 1 is properly positioned over the puncture wound site.

Device 1 is preferably formed by injection molding of a resilient thermoplastic polymer, although other equivalent materials and methods may be used.

Although it is preferred to employ the ratcheting mechanism herein disclosed as permitting only unidirectional movement of plunger 4 in cylinder 3, a screw machine (not shown herein) or other similar mechanisms may be employed.

The method of using device 1 to achieve hemostasis at the site of a puncture wound will now be described.

In practice, after the removal of the needle from the puncture site in the patient's arm, device 1 is positioned over the said site, apertures 14 in footplate 2 permitting visual observance by the medical technician to insure that rounded portion 10 of plunger 4 and compression pad 11 adhered thereto are accurately placed over the puncture wound site.

Adhesive pads 12 securely hold device 1 in position on skin 13. The index finger of the medical technician is placed on arcuate elements 9 of plunger 4. The thumb of the medical technician is placed in engagement with one of the curved arms 5 and the middle finger of the medical technician is placed in engagement with the other of the curved arms 5. The said index finger is now used to force down plunger 4 until compression pad 11 firmly bears against the puncture wound, the said thumb and middle finger of the medical technician in clamping engagement with curved arms 5 holding device 1 firmly in position until hemostasis is achieved. Because plunger 4 is prevented from moving away from the puncture wound due to the ratcheting effect between cylinder 3 and plunger 4, the index finger of the medical technician may be removed from arcuate elements 9 of plunger 4.

After hemostasis has been achieved, device 1 can be removed from the skin 13 of the patient, and a surgical dressing may then be applied to the site of the puncture wound.

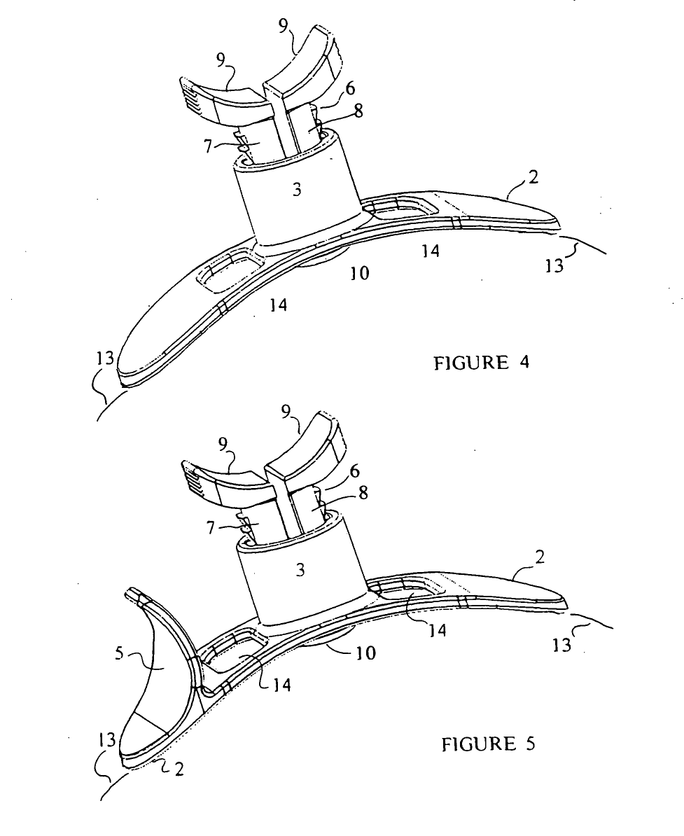

In the alternate embodiment shown in FIG. 4, curved arms 5 have been dispensed with. The thumb and middle finger of the medical technician engage cylinder 3 on opposite sides thereof and function just as they did in the embodiment of FIGS. 1-3.

In the alternate embodiment shown in FIG. 5, only one curved arm 5 is employed. The thumb and middle finger of the medical technician engage curved arm 5 and this side of cylinder 3 opposite the said curved arm 5. In this embodiment, the thumb and middle finger of the medical technician function just as they did in the embodiment shown in FIGS. 1-3.

Although I have described my invention in considerable detail, modifications and changes which do not depart from the spirit of the invention as shown herein may occur to those skilled in the art to which this invention pertains, the appended claims should be construed as covering such modifications and equivalents as are suitable to the practice of the invention.

I claim:

1. A device for use by a medical technician to achieve hemostasis at the site of a puncture wound in the skin of a patient, said device comprising:
(a) a footplate,
(b) a cylinder on said footplate,
(c) a plunger positioned in said cylinder and movable therethrough and through said footplate, the top of said plunger being engageable by the index finger of said medical technician,
(d) motion restricting means interposed between said plunger and the interior of said cylinder, said motion restricting means permitting said plunger to be moved only toward said puncture wound site and preventing movement of said plunger away from said puncture wound site,
(e) stabilizing means engageable with the thumb and middle finger of the medical technician,
(f) whereby the thumb and middle finger of the medical technician may be engaged with said stabilizing means to firmly hold said footplate in position on the skin of the patient with the plunger located over the said puncture wound site, and the index finger of the medical technician is engageable with the top of the plunger, whereupon the medical technician may force the plunger to advance through the cylinder and footplate to press against the site of the puncture wound.

2. A device as in claim 1, wherein:
(g) said stabilizing means comprises two arms positioned adjacent opposite sides of said cylinder.

3. A device as in claim 1, wherein:
(g) said stabilizing means comprises two opposite sides of said cylinder.

4. A device as in claim 1, wherein:
(g) said stabilizing means comprises:
(i) one arm on said footplate adjacent one side of said cylinder,
(ii) the side of said cylinder opposite said one arm.

5. A device for use by a medical technician to achieve hemostasis at the site of a puncture wound in the skin of a patient, said device comprising:
(a) a footplate,
(b) a cylinder centrally located on said footplate,
(c) a plunger positioned in said cylinder and movable therethrough and through said footplate toward said site, the top of said plunger being engageable by the index finger of said medical technician,
(d) motion restricting means interposed between said plunger and the interior of said cylinder, said motion restricting means permitting said plunger to be moved only toward said site and preventing movement of said plunger away from said site,
(e) a first arm extending upwardly on said footplate adjacent one side of said cylinder and engageable by the thumb of said medical technician,
(f) a second arm extending upwardly on said footplate adjacent the opposite side of said cylinder and engageable by the middle finger of said medical technician,
(g) whereby, in use, the thumb and middle finger of the medical technician are engaged with said first and second arms respectively to firmly hold said footplate in position on the skin of said patient with the plunger located over the said site, and the index finger of the medical technician is engaged with the top of the plunger, whereupon the medical technician forces the plunger to advance through the cylinder and footplate to press against the site of the puncture wound.

6. A device as in claim 5, wherein:
(h) said motion restricting means comprises:
(i) external ratchets on said plunger,
(ii) internal ratchets in aid cylinder and engageable with said external ratchets,
(iii) said external and internal ratchets cooperating to permit only unidirectional movement of said plunger toward said site.

7. A device as in claim 5, further comprising:
(h) a compression pad adhered to the bottom of said plunger and engageable with the skin of the patient at said site when the device is in use.

8. A device as in claim 5, wherein:
(h) the top of said plunger is curved to engage the index finger of the medical technician.

9. A device as in claim 5, wherein:
(h) the upper portion of said plunger is bifurcated, each of said bifurcations being curved to engage the index finger of the medical technician.

10. A device as in claim 5, further comprising:
(h) a pad having a first surface and a second surface opposite said first surface,
(i) an adhesive on said first surface engageable with the bottom of said footplate to secure said pad to said footplate,
(j) an adhesive on the second surface of said pad to firmly secure said device in position on the skin of the patient.

11. A device as in claim 5, further comprising:
(h) an aperture through said footplate exposing the skin of the patient to observation by the medical technician, whereby said medical technician can accurately position said plunger over the site of the puncture wound.

12. A device for use by a medical technician to achieve hemostasis at the site of a puncture wound in the skin of a patient, said device comprising:
(a) a footplate,
(b) a cylinder centrally located on said footplate,
(c) a plunger positioned in said cylinder and movable therethrough and through said footplate toward said site, the top of said plunger being engageable by the index finger of said medical technician,
(d) motion restricting means interposed between said plunger and the interior of said cylinder, said motion restricting means permitting said plunger to be moved only toward said puncture wound site and preventing movement of said plunger away from said puncture wound site,
(e) whereby, in use, the thumb and middle finger of said medical technician are engaged with opposite sides of said cylinder to firmly hold said footplate in position on the skin of said patient with the plunger located over the said site, and the index finger of said medical technician is engaged with the top of said plunger, whereupon the medical technician may force the plunger to advance through the cylinder and footplate to press against the site of the puncture wound.

13. A device as in claim 12, wherein:
(f) said motion restricting means comprises:
(i) external ratchets on said plunger,
(ii) internal ratchets in said cylinder and engageable with said external ratchets,
(iii) said external and internal ratchets cooperate to permit only unidirectional movement of said plunger toward said site.

14. A device as in claim 12, further comprising:
(f) a compression pad adhered to the bottom of said plunger and engageable with the skin of the patient at said site when the device is in use.

15. A device as in claim 12, wherein:
(f) the top of said plunger is curved to engage to engage the index finger of the medical technician.

16. A device as in claim 12, wherein:
(f) the upper portion of said plunger is bifurcated, each of said bifurcations being curved to engage the index finger of the medical technician.

17. A device as in claim 12, further comprising:
(f) a pad having a first surface and a second surface opposite said first surface,
(g) an adhesive on said first surface engageable with the bottom of said footplate to secure said pad to said footplate,
(h) an adhesive on the second surface of said pad to firmly secure said device in position on the skin of the patient.

18. A device as in claim 17, further comprising:
(f) an aperture through said footplate exposing the skin of the patient to observation by said medical technician, whereby said medical technician can accurately position said plunger over the site of the puncture wound.

19. A device for use by a medical technician to achieve hemostasis at the site of a puncture wound in the skin of a patient, said device comprising:
(a) a footplate,
(b) a cylinder centrally located on said footplate,
(c) a plunger positioned in said cylinder and movable therethrough and through said footplate toward said site, the top of said plunger being engageable by the index finger of said medical technician,
(d) motion restricting means interposed between said plunger and the interior of said cylinder, said motion restricting means permitting said plunger to be moved only toward said site and preventing movement of said plunger away from said site,
(e) an arm extending upwardly on said footplate adjacent one side of said cylinder and engageable by one finger of the medical technician,
(f) whereby, in use, the said one finger of the medical technician is engaged with said arm, another finger on the same hand of the medical technician is engaged with that side of said cylinder opposite said arm, the two said fingers firmly holding said footplate in position on the skin of the patient with the plunger located over the said site, and the index finger of the medical technician is engaged with the top of the plunger, whereupon the medical technician forces the plunger to advance through the cylinder and footplate to press against the site of the puncture wound.

20. A device as in claim 19, wherein:
(g) said motion restricting means comprises:
(i) external ratchets on said plunger,
(ii) internal ratchets in said cylinder and engageable with said external ratchets,
(iii) said external and internal ratchets cooperate to permit only unidirectional movement of said plunger toward said site.

21. A device as in claim 19, further comprising:
(f) a compression pad adhered to the bottom of said plunger and engageable with the skin of the patient at said site when the device is in use.

22. A device as in claim 19, wherein:
(f) the top of said plunger is curved to engage the index finger of the medical technician.

23. A device as in claim 19, wherein:
(f) the upper portion of said plunger is bifurcated, each of said bifurcations being curved to engage the index finger of the medical technician.

24. A device as in claim 19, further comprising:
(f) a pad having a first surface and a second surface opposite said first surface,
(g) an adhesive on said first surface engageable with the bottom of said footplate to secure said pad to said footplate,
(h) an adhesive on the second surface of said pad to firmly secure said device in position on the skin of the patient.

25. A device as in claim 19, further comprising:
(f) an aperture through said footplate exposing the skin of the patient to observation by said medical technician, whereby said medical technician can accurately position said plunger over the site of the puncture wound.
